(19) United States
(12) Patent Application Publication
(10) Pub. No.: US 2026/0174655 A1
(43) Pub. Date: Jun. 25, 2026

(54) SOAP COMPOSITIONS

(71) Applicant: HENKEL AG & CO. KGAA, Düsseldorf (DE)

(72) Inventor: Joseph D. DAHLMEYER, JR., Hamden, CT (US)

(21) Appl. No.: 19/408,186

(22) Filed: Dec. 3, 2025

Related U.S. Application Data
(60) Provisional application No. 63/737,336, filed on Dec. 20, 2024.

(51) Int. Cl. A61K 8/44; A61K 8/27; A61K 8/36; A61K 8/41; A61K 8/42; A61Q 19/10

(52) U.S. Cl. CPC A61K 8/442; A61K 8/27; A61K 8/36; A61K 8/416; A61K 8/42; A61Q 19/10; A61K 2800/48; A61K 2800/596

(57) ABSTRACT

Soap compositions useful for cleaning applications are described. The soap compositions include one or more salts that lower the zein score of the soap composition signifying a milder composition. The soap compositions can also include at least one non-ionic surfactant, at least one cationic surfactant, and at least one amphoteric or zwitterionic surfactant. The one or more salts can be in an amount from about 0.01 wt % to about 1.0 wt % of the soap composition.

### CROSS REFERENCE TO RELATED APPLICATION

This application claims the benefit of U.S. Provisional Application No. 63/737,336, filed on Dec. 20, 2024, the entire contents of which are incorporated by reference herein.

### BACKGROUND

Mildness is an important attribute for hand soap formulations, especially considering the frequency at which these formulations contact a consumer's skin. Zein is a corn-based protein that has similar chemistry to proteins found in the outer layer of skin. These proteins, when interacted upon by surfactants, can denature (unfold) and lead to moisture loss or irritation of the skin. The extent to which surfactant-based formulations denature this protein can be used as an indicator of the mildness (or harshness) of the formulation. Zein score (amount of denatured zein protein) can therefore be used to compare the mildness of prototype formulations and can be used to formulate new products with improved mildness versus commercial surfactant-based systems.

There is a need for compositions that have a lower zein score compared to what is currently available to the consumer.

### SUMMARY OF THE INVENTION

In general, disclosed herein are new soap compositions and methods of making the same. Various non-limiting inventive aspects according to the disclosure are as follows.

In some instances, a first aspect of the disclosure can be described as a soap composition comprising, consisting essentially of, or consisting of at least one non-ionic surfactant, at least one cationic surfactant, at least one amphoteric or zwitterionic surfactant, and at least one salt.

In some instances, a second aspect of the disclosure can be described as a soap composition according to the first aspect, wherein the at least one salt is in an amount ranging from about 0.01 wt % to about 1.0 wt % of the soap composition.

In some instances, a third aspect of the disclosure can be described as a soap composition according to the first or second aspect wherein soap composition has a cationic surfactant(s): amphoteric/zwitterionic surfactant(s): non-ionic surfactant(s) molar percentage (mol %) ratio ranging from 0.1-75.0:5.0-99.0:0.1-40.0.

In some instances, a fourth aspect of the disclosure can be described as a soap composition according to any one of the first through third aspects, wherein the at least one non-ionic surfactant is an alkanolamide.

In some instances, a fifth aspect of the disclosure can be described as a soap composition according to the fourth aspect, wherein the alkanolamide is cocamide monoethanolamine (CMEA).

In some instances, a sixth aspect of the disclosure can be described as a soap composition according to any one of the first through fifth aspects, wherein the at least one cationic surfactant is an ammonium salt.

In some instances, a seventh aspect of the disclosure, can be described as a soap composition according to the sixth aspect, wherein the ammonium salt is a quaternary ammonium salt.

In some instances, an eighth aspect of the disclosure, can be described as a soap composition according to the seventh aspect, wherein the quaternary ammonium salt is cetyl trimethyl ammonium chloride (CTAC).

In some instances, a ninth aspect of the disclosure can be described as a soap composition according to any one of the first through eighth aspects, wherein the at least one amphoteric or zwitterionic surfactant is a betaine.

In some instances, a tenth aspect of the disclosure can be described as a soap composition according to the ninth aspect, wherein the betaine is cocamidopropyl betaine (CAPB).

In some instances, an eleventh aspect of the disclosure can be described as a soap composition according to any one of the first through tenth aspects, wherein the at least one salt is sodium benzoate, zinc sulfate, sodium ethylenediamine-N,N′-disuccinate (Na-EDDS), or any combination thereof.

In some instances, a twelfth aspect of the disclosure can be described as a soap composition according to any one of the first through eleventh aspects, wherein the soap composition has a viscosity ranging from about 1 cps to about 6,000 cps.

In some instances, a thirteenth aspect of the disclosure can be described as a soap composition according to any one of the first through twelfth aspects, wherein the soap composition has a viscosity ranging from about 1,000 cps to about 3,000 cps.

In some instances, a fourteenth aspect of the disclosure can be described as a soap composition according to any one of the first through twelfth aspects, wherein the soap composition has a viscosity ranging from about 1 cps to about 1,000 cps.

In some instances, a fifteenth aspect of the disclosure can be described as a soap composition according to any one of the first through fourteenth aspects, wherein the soap composition has a Zein score of less than 3.0%.

In some instances, a sixteenth aspect of the disclosure can be described as a soap composition according to any one of the first through fifteenth aspects, wherein the soap composition has a Zein score of less than 2.0%.

In some instances, a seventeenth aspect of the disclosure can be described as a soap composition according to any one of the first through sixteenth aspects, wherein the soap composition has a Zein score of less than 1.0%.

In some instances, an eighteenth aspect of the disclosure can be described as a soap composition according to any one of the first through seventeenth aspects, wherein the soap composition has a pH ranging from about 2 to about 7.

In some instances, a nineteenth aspect of the disclosure can be described as a soap composition according to any one of the first through seventeenth aspects, wherein the soap composition has a pH ranging from about 7 to about 10.

In some instances, a twentieth aspect of the disclosure can be described as a soap composition according to any one of the first through nineteenth aspects, wherein the soap composition further comprises a rheology modifier.

In some instances, a twenty-first aspect of the disclosure can be described as a soap composition according to the twentieth aspect, wherein the rheology modifier is a thickener.

In some instances, a twenty-second aspect of the disclosure can be described as a soap composition according to any one of the first through twenty-first aspects, wherein the soap composition is a hand soap composition.

In some instances, a twenty-third aspect of the disclosure can be described as a hand soap comprising, consisting essentially of, or consisting of a soap composition according to any one of the first through twenty-first aspects.

In some instances, a twenty-fourth aspect of the disclosure can be described as a use of a hand soap according to the twenty-third aspect to clean hands.

### BRIEF DESCRIPTION OF DRAWINGS

FIG. 1 is a ternary plot of ratios of cetyltrimethylammonium chloride (CTAC), cocamidopropyl betaine (CAPB), and cocamide monoethanolamine (CMEA) used in different hand soap formulations according to various aspect of the present disclosure.

FIG. 2 is a ternary plot with a viscosity line representing the minimum required % of CMEA to achieve at least 3000 cps.

FIG. 3 is an image of the “Response Optimizer” tool in the JMP software that can be used to determine the viscosity.

FIG. 4 is bar graph of the zein score data of different systems with and without salts.

FIG. 5 is a ternary plot of the ratios of CTAC, CAPB, and CMEA used in different hand soap formulations according to various aspect of the present disclosure, where the red/shaded region (containing data points therein) indicates compositions with higher zein scores than a benchmark system “P”. The unshaded region (also containing data points) indicates the range of CTAC/CAPB/CMEA blends that yield lower zein scores than the benchmark.

### DETAILED DESCRIPTION

The following description of the embodiments is merely exemplary in nature and is in no way intended to limit the subject matter of the present disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the terms “about” or “approximately.” The use of the terms “about” and “approximately” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, alternatively ±1 percent, alternatively ±0.5 percent, and alternatively ±0.1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Various aspects of the present disclosure are directed towards soap compositions and methods of using the same. In accordance with various aspects of the disclosure, certain soap compositions comprise or consist essentially of a salt, at least one cationic surfactant, at least one amphoteric or zwitterionic surfactant, and at least one non-ionic surfactant.

Cationic surfactants are known anti-microbial agents but have the detriment of harshness to the skin (harshness defined as high zein scores). In accordance with various aspects of the disclosure, however, the inventors have discovered the use of salts in soap compositions having cationic surfactants results in soap compositions having zein scores that are lower, and thus milder to a user, than soap compositions that do not comprise a combination of cationic surfactant(s) and salt(s). Without being bound to any particular theory, the inventors believe that the use of salts results in electrostatic screening of the positive charge of cationic surfactants, creating a pseudo-nonionic surfactant. This shift in surfactant charge from a positive charge towards a neutral charge has been observed to cause an increase in the mildness (decrease in zein score) of surfactant systems containing cationic surfactant(s) that are otherwise considered harsh on a user's skin.

The inventors have further discovered that, in some instances, the use of salts in soap compositions according to various aspects of the disclosure not only results in a lowered zein score, but also an increase in the viscosity of the soap composition, which is beneficial for the preparation of gel or gel-like soap compositions. As such, the incorporation of one or more salts, as described elsewhere herein, also provides a means of modifying the viscosity of soap compositions according without requiring any additional rheology modifiers.

The inventors have further discovered that, in some instances, the use of additional rheology modifiers in soap compositions containing one or more salts does not detrimentally affect the zein score of the soap composition (which as discussed above, is lower than soap compositions that do not comprise a combination of cationic surfactant(s) and salt(s)). As such, soap compositions according to various aspects of the disclosure that exhibit desirable properties with respect to skin irritability (a low zein score) but undesirable properties with respect to mode of user application (as, e.g., a liquid, a gel, a viscoelastic solid) can be modified to achieve a target viscosity by the addition of rheology modifiers without loss of mildness.

Various cationic surfactants may be used in soap compositions according to various aspects of the disclosure. In some instances, cationic surfactants are ammonium salt surfactants having a structure according to Formula (I):

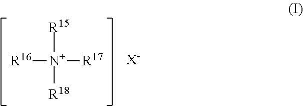

wherein R¹⁵ is a linear or branched alkyl, aryl, or alkylaryl group having from about 12 to about 30 carbon atoms, each of R¹⁶, R¹⁷, and R¹⁸ is, independently, hydrogen, linear or branched alkyl, aryl, or alkylaryl group having from about 12 to about 22 carbon atoms, and X is a counter anion. In some instances, the counter anion is chloride, bromide, iodide, acetate, phosphate, nitrate, sulfate, methyl sulfate, ethyl sulfate, tosylate, lactate, citrate, glycolate, or a combination thereof. In some instances, R¹⁵ and optionally one or more of R¹⁶, R¹⁷, and R¹⁸ can contain one or more heteroatoms (for example, O, N, and S) to provide for, for example, an ester group, an ether group, a sulfide group, a hydroxyl group, an amine group (an so on) incorporated in the chemical structure thereof. In some instances, R¹⁵ and optionally one or more of R¹⁶, R¹⁷, and R¹⁸ can contain, for example polyethylene glycol and/or polypropylene glycol groups.

In some instances, R¹⁵ is a branched or linear alkyl group having about 12 to about 22 carbon atoms, R¹⁶ is H or a branched or linear alkyl group having 1 to about 22 carbon atoms, R¹⁷ is H or an alkyl group having 1 to about 3 carbon atoms, and R¹⁸ is H or an alkyl group having 1 to about 3 carbon atoms. In some instances, R¹⁵ is a branched or linear alkyl group having about 12 to about 22 carbon atoms, and each of R¹⁶, R¹⁷, and R¹⁸ are H or alkyl groups having 1 to about 3 carbon atoms.

In some instances, soap compositions according to various aspects of the disclosure include at least one ammonium salt surfactant according to Formula (I), where the at least one ammonium salt surfactant is a primary ammonium salt (that is, each of R¹⁶, R¹⁷, and R¹⁸ is H). In some instances, soap compositions according to various aspects of the disclosure include at least one ammonium salt surfactant according to Formula (I), where the at least one ammonium salt surfactant is a secondary ammonium salt (that is, only two of R¹⁶, R¹⁷, and R¹⁸ are H). In some instances, soap compositions according to various aspects of the disclosure include at least one ammonium salt surfactant according to Formula (I), where the at least one ammonium salt surfactant is a tertiary ammonium salt (that is, only one of R¹⁶, R¹⁷, and R¹⁸ are H). In some instances, soap compositions according to various aspects of the disclosure include at least one ammonium salt surfactant according to Formula (I), where the at least one ammonium salt surfactant is a quaternary ammonium salt (that is, none of R¹⁶, R¹⁷, and R¹⁸ are H). Non-limiting examples of ammonium salt surfactants for soap compositions according to various aspects of the disclosure include, but are not limited to, cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, or a combination thereof. In some instances, the use of cetyl trimethyl ammonium chloride (CTAC) as the at least one ammonium salt surfactant is preferred.

In some instances, the ammonium salt surfactants suitable for use in soap compositions according to the disclosure include those where at least one functional (or R) group of the ammonium salt surfactant is derived from a tallow fatty acid or a coconut fatty acid. The term “tallow” refers to an alkyl group derived from tallow fatty acids (usually hydrogenated tallow fatty acids), which generally has mixtures of alkyl chains in the C₁₆-C₁₈ range. The term “coconut” refers to an alkyl group derived from a coconut fatty acid, which generally have mixtures of alkyl chains in the C₁₂-C₁₄ range. Non-limiting examples of ammonium salt surfactants derived from tallow fatty acids or coconut fatty acids include, but are not limited to, ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, or any combination thereof.

In some instances, soap compositions according to various aspect of the disclosure may include one or more cationic surfactants that are ammonium salt surfactants having a structure according to Formula (I) where R¹⁶-R¹⁸ are as described above and where R¹⁵ is R¹⁹—C(═O)—NH—(CH₂)ₙ—, where R¹⁹ is a branched or linear alkyl group having from about 12 to about 22 carbon atoms, and n is an integer ranging from 2 to 6, alternatively n is an integer ranging from 2 to 4, and alternatively n is an integer of 2 or 3. Non-limiting examples such ammonium salt surfactants include, but are not limited to, stearamidopropyl propylene glycol (PG)-dimonium chloride phosphate, behenamidopropyl PG-dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

In some instances, soap compositions according to various aspects of the disclosure may include at least one non-ionic surfactant. Various non-ionic surfactants may be used in soap compositions according to the disclosure. In some instances, suitable nonionic surfactants include, but are not limited to, alkanolamides. alkoxylated alcohols, polyoxyalkylene alkyl ethers, polyoxyalkylene alkylphenyl ethers, polyoxyalkylene sorbitan fatty acid esters, polyoxyalkylene sorbitol fatty acid esters, polyalkylene glycol fatty acid esters, alkyl polyalkylene glycol fatty acid esters, polyoxyethylene polyoxypropylene alkyl ethers, polyoxyalkylene castor oils, polyoxyalkylene alkylamines, glycerol fatty acid esters, alkylglucosamides, alkylglucosides, alkylamine oxides, or any combinations thereof. In some instances, the use of an alkanolamide as a non-ionic surfactant is preferred. In some instances, the use of cocamide monoethanolamine (CMEA) as an alkanolamide non-ionic surfactant is preferred.

In some instances, soap compositions according to various aspects of the disclosure may include at least one amphoteric or zwitterionic surfactant. Various non-ionic surfactants may be used in soap compositions according to the disclosure. In some instances, suitable amphoteric or zwitterionic surfactants include, but are not limited to, alkylamidopropylamine N-oxides (APAOs), alkyldimethylamine N-oxides (AOs), alkylbetaines (ABts), alkylamidopropylbetaines (APBs), cocamidopropyl betaine, cocoamphoacetate, cocoamphodiacetate, lauryl betaine, betaine citrate, sodium lauroamphoacetate, cocoamine, imidazolidine, or sodium hydroxymethylglycinate. In some instances, the amphoteric or zwitterionic surfactant is cocamidopropyl betaine (CAPB).

Soap compositions according to various aspects of the disclosure include at least one salt. Various salts may be used in soap compositions according to the disclosure. In some instances, the salt is an alkali metal salt (such as sodium or potassium salts), an alkaline earth metal salt (such as magnesium or calcium salts), an ammonium salt, an iron salt, a zinc salt, or an aluminum salt, or any combination thereof. In some instances, the salt is a sulfate salt, a carboxylate (e.g., acetates, citrates, acrylates, benzoates, succinates such as ethylenediamine-N,N′-disuccinate) salts, chloride salts, carbonate salts, nitrate salts, phosphate salts, and any combination thereof. In some instances, suitable salts include, but are not limited to, sodium benzoate, zinc sulfate and sodium ethylenediamine-N,N′-disuccinate (Na-EDDS), sodium chloride, and magnesium sulfate (Epsom salt). In some instances, the use of a combination of at least two of sodium benzoate, zinc sulfate and Na-EDDS is preferred. In some instances, soap compositions according to the disclosure comprise a total salt(s) content ranging from about 0.01 to about 5 wt %, relative to the total weight of the soap composition. In some instances, soap compositions according to the disclosure comprise a total salt(s) content ranging from about 0.025 to about 4 wt %, alternatively from about 0.05 to about 3 wt %, alternatively from about 0.075 to about 3 wt %, alternatively from about 0.1 to about 2 wt %, and alternatively from about 0.1 to about 1 wt %, relative to the total weight of the soap composition.

In some instances, soap compositions according to the disclosure may further include at least one rheology modifier. Various rheology modifier may be used in soap compositions according to the disclosure to either increase or decrease the viscosity of the soap composition. In some instances, the at least one rheology modifier is a thickener. In some embodiments, the use of Glucamate DOE-120 (PEG-120 methyl glucose dioleate thickening polymer) as a thickener is preferred.

Soap compositions according to the disclosure may further comprise water. The amount of water used in a soap composition according to the disclosure may vary. In some instances, soap compositions according to the disclosure may comprise a total water content ranging from about 60 to about 99 wt %, relative to the total weight of the soap composition. In some instances, soap compositions according to the disclosure comprise a total water content ranging from about 65 to about 98 wt %, alternatively from about 70 to about 97 wt %, alternatively from about 75 to about 96 wt %, alternatively from about 80 to about 95 wt %, alternatively from about 80 to about 95 wt %, relative to the total weight of the soap composition.

Soap compositions according to the disclosure may have varying amounts of the least one cationic surfactant, the at least one amphoteric or zwitterionic surfactant, and the at least one non-ionic surfactant. In some instances, soap composition according to the disclosure are prepared such that they have a cationic surfactant(s): amphoteric/zwitterionic surfactant(s): non-ionic surfactant(s) molar percentage (mol %) ratio ranging from 0.1-75.0:5.0-99.0:0.1-40.0. In some instances, soap compositions according to the disclosure may comprise a total surfactants content (that is, the total combined amount the least one cationic surfactant, the at least one amphoteric or zwitterionic surfactant, and the at least one non-ionic surfactant in a soap composition) ranging from about 1 to about 25 wt %, relative to the total weight of the soap composition. In some instances, soap compositions according to the disclosure comprise a total surfactants content ranging from about 2.5 to about 20 wt %, alternatively from about 5 to about 15 wt %, and alternatively from about 7.5 to about 12.5 wt % relative to the total weight of the soap composition.

Soap compositions according to various aspects of the disclosure may be prepared to have different viscosities. In some instances, the viscosity of soap compositions according to the disclosure is between 1 cps and 10,000 cps. In some instances, the viscosity of soap compositions according to the disclosure is between 1 cps and 8,000 cps. In some instances, the viscosity of soap compositions according to the disclosure is between 1 cps and 6,000 cps. In some instances, the viscosity of soap compositions according to the disclosure is between 100 cps and 6,000 cps. In some instances, the viscosity of soap compositions according to the disclosure is between 500 and 6,000 cps. In some instances, the viscosity of soap compositions according to the disclosure is between 1,000 and 6,000 cps. In some instances, the viscosity of soap compositions according to the disclosure is between 2,000 and 6,000 cps. In some instances, the viscosity of soap compositions according to the disclosure is between 3,000 and 6,000 cps. In some instances, the viscosity of soap compositions according to the disclosure is between 500 and 5,000 cps. In some instances, the viscosity of soap compositions according to the disclosure is between 500 and 4,500 cps. In some instances, the viscosity of soap compositions according to the disclosure is between 500 and 4,000 cps. In some instances, the viscosity of soap compositions according to the disclosure is between 500 and 3,500 cps. In some instances, the viscosity of soap compositions according to the disclosure is between 1,000 and 3,000 cps. In some instances, the viscosity of soap compositions according to the disclosure is between 1 and 20 cps. In some instances, the viscosity of soap compositions according to the disclosure is between 1 and 50 cps. In some instances, the viscosity of soap compositions according to the disclosure is between 1 and 100 cps. In some instances, the viscosity of soap compositions according to the disclosure is between 1 and 250 cps. In some instances, the viscosity of soap compositions according to the disclosure is between 1 and 500 cps. In some instances, the viscosity of soap compositions according to the disclosure is between 1 and 750 cps. In some instances, the viscosity of soap compositions according to the disclosure is between 1 and 1,000 cps. In some instances, the viscosity of a soap composition according to the disclosure is such that the soap composition is in the form of a liquid. In some instances, the viscosity of a soap composition according to the disclosure is such that the soap composition is in the form of a gel or gel-like composition. In some instances, the viscosity of a soap composition according to the disclosure is such that the soap composition exhibits properties indicative of a viscoelastic solid.

Soap compositions according to various aspects of the disclosure can have pH values ranging from about 2 to about 10. In some instances, soap compositions according to the disclosure can have pH values ranging about 3 to about 9, alternatively from about 3 to about 7, alternatively from about 4 to about 7, and alternatively from about 4 to about 6. In some instances, soap compositions according to the disclosure can have pH values ranging from about 2 to about 7. In some instances, soap compositions according to the disclosure can have pH values ranging from about 7 to about 10.

One method to test the skin irritancy potential of a surfactant composition is Zein test. Zein score is measured using a Zein test (Gott, E., Aesthet. Medzin., Tenside 15:313 (1966)). Zein test determines the extent of denaturation of Zein corn protein after exposure to a surfactant for a given period of time. Generally, the higher the Zein score, the greater the skin irritation potential.

As used herein the phrase “Zein score” refers the measurement obtained from the Zein test as described herein. It can also be referred to as “Zein solubilized %.” In some instances, soap compositions described herein have a Zein score of less than about 3% when tested at a 10% dilution. In certain instances, soap compositions according to the disclosure have a Zein score of from about 0.01% to about 3%, alternatively from about 0.01% to about 2.75%, alternatively from about 0.01% to about 2.5%, alternatively from about 0.01% to about 2.25%, alternatively from about 0.01% to about 2%, alternatively from about 0.01% to about 1.75%, alternatively from about 0.01% to about 1.5%, alternatively from about 0.01% to about 1.25%, and alternatively from about 0.01% to about 1% when tested at a 10% dilution. In other instances, soap compositions according to the disclosure can have a Zein score of less than about 2.75%, preferably less than about 2.5%, more preferably less than about 2.25%, even more preferably less than about 2%, even more preferably less than 1.75%, even more preferably less than about 1.5%, even more preferably less than about 1.25%, and even more preferably less than about 1% when tested at a 10% dilution. In some instances, soap compositions according to the disclosure have a Zein score less than 0.5%. In some instances, soap compositions according to the disclosure have a Zein score ranging from about 0.3 to about 0.4%.

Another method to measure the skin irritancy potential is corneosurfametry (CSM) test, a noninvasive quantitative test that measures the interaction between surfactants and human stratum corneum (Pierard et al., Dermatology 189:152-156 (1994)). Corneosurfametry involves removing a few layers of skin using cyanoacrylate skin surface strippings, short contact time with surfactants followed by staining the samples with fuchian dyes. A less damaged barrier allows greater penetration of the stain, therefore giving a more intense color, which is measured using colorimetrically with L*a*b*color space. This method is predictive of both protein and lipid damage in the skin. CIM (Color Indicator of Mildness) values are obtained from a corneosurfametry test. In a comparative study, the higher the CIM value, the milder the surfactant formulation.

A third method to evaluate the irritation effect of a surfactant formulation is measured by cytokine release of representative human skin model in response to the surfactant formulation. Where skin tissue viability is not decreased by 50% as compared to the negative control tissue (as measured by MTT reduction), the inflammatory potential is then measured by the production of the cytokines IL-la. and/or IL-1ra. MTT is a dye used to stain the skin cells called 3-(4,5-dimethylthiazol-2-yl)-2,5-diphenyltetrazolium bromide. In a comparative study, a lower cytokine release value means a milder surfactant formulation.

Described in US 2018/0016523, the entire contents of which are incorporated by reference herein, are methods for determining how mild an aqueous soap formulation is for a user. The method comprises the steps of: (i) providing a solution comprising a laundry cleaning effective amount of a surfactant composition, (ii) subjecting an appropriate dilution of the solution to Zein test, corneosurfametry test, and in vitro cytokine release test for IL-la and IL-1ra response, respectively, to obtain a Zein score, a corneosurfametric (CSM) value, and a cytokine release value, and (iii) deriving at composite mildness indicator (CMI) for each solution based the Zein score, the CSM value, and the cytokine release value.

The composite mildness indicator (CMI) may also be called detergent mildness indicator (DMI). Specifically, the CMI can be derived from (1) the Zein score, CSM value, and cytokine release value IL-la of the formulation; (2) the Zein score, CSM value, and cytokine release value (IL-1a+IL-1ra) of the formulation; (3) the Zein score, CSM value, and cytokine release value (IL-1ra/IL-la) of the formulation; (4) the Zein score, CSM value, and cytokine release value log 10 (IL-1ra/IL-la) of the formulation; or (5) the Zein score, CSM value, and cytokine release measurement (IL-la/IL-1ra) of the formulation.

### Examples

In accordance with various aspects of the present disclosure presents the ability to increase the concentration of cationic surfactant (CTAC) in hand soaps by replacing the two amine oxides in a current commercial antibacterial hand soap with a combination of cocamidopropyl betaine and cocamide MEA, as shown below in Table 1.

TABLE 1

| Comparative Prior Art Hand Soap | Soap Compositions According to the |
|---|---|
| Composition | Invention |
| CTAC | CTAC |
| Lauramine oxide (e.g., Ammonyx LO) | Cocamidopropyl Betaine (CAPB) |
| Lauryl/myristyl amidoamine oxide (e.g., | Cocamide monoethanolamine (CMEA) |
| Ammonyx LMDO) | |

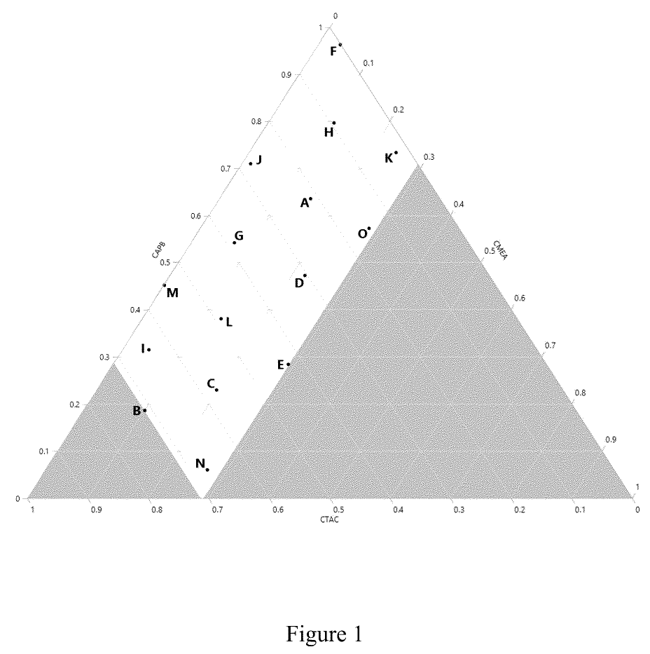

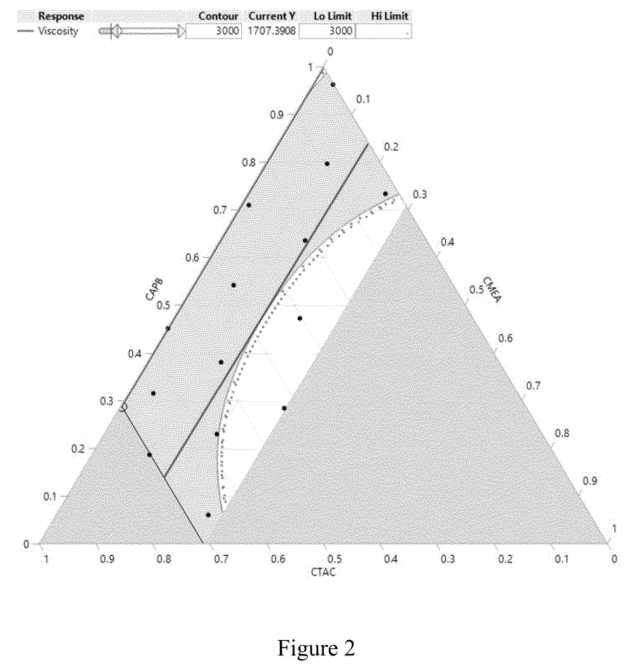

A Space Filling Mixture DOE (or Design Of Experiments) was constructed to examine different ratios of the three surfactants, CTAC, CAPB, and CMEA in soap compositions according to the disclosure. A ternary plot shows comparing the different ratios of these surfactants is displayed in FIG. 1. Tables 2 and 3 provide the corresponding surfactant ratios and surfactant % actives as displayed FIG. 1. FIG. 2 is the same ternary plot as FIG. 1, but a line indicating the minimum required % CMEA to achieve at least 3000 cps viscosity is provided.

TABLE 2

| | DOE/Surfactant | | | | | Active % (after post doses) | | | |
|---|---|---|---|---|---|---|---|---|---|
| | CTAC | CAPB | CMEA | Total | | CTAC | CAPB | CMEA | Total |
| A | 0.214 | 0.636 | 0.150 | 1.000 | A | 0.912 | 2.710 | 0.641 | 4.264 |
| B | 0.713 | 0.186 | 0.101 | 1.000 | B | 3.039 | 0.794 | 0.431 | 4.264 |
| C | 0.572 | 0.230 | 0.198 | 1.000 | C | 2.441 | 0.979 | 0.845 | 4.264 |
| D | 0.305 | 0.472 | 0.223 | 1.000 | D | 1.300 | 2.014 | 0.949 | 4.264 |
| E | 0.427 | 0.284 | 0.290 | 1.000 | E | 1.820 | 1.210 | 1.234 | 4.264 |
| F | 0.001 | 0.963 | 0.036 | 1.000 | F | 0.006 | 4.104 | 0.154 | 4.264 |
| G | 0.387 | 0.542 | 0.071 | 1.000 | G | 1.649 | 2.311 | 0.304 | 4.264 |
| H | 0.095 | 0.796 | 0.109 | 1.000 | H | 0.404 | 3.396 | 0.463 | 4.264 |
| I | 0.642 | 0.315 | 0.044 | 1.000 | I | 2.736 | 1.342 | 0.186 | 4.264 |
| J | 0.276 | 0.710 | 0.014 | 1.000 | J | 1.177 | 3.026 | 0.061 | 4.264 |
| K | 0.024 | 0.734 | 0.243 | 1.000 | K | 0.101 | 3.128 | 1.035 | 4.264 |
| L | 0.489 | 0.381 | 0.130 | 1.000 | L | 2.087 | 1.623 | 0.554 | 4.264 |
| M | 0.548 | 0.452 | 0.001 | 1.000 | M | 2.334 | 0.257 | 0.004 | 4.264 |
| N | 0.672 | 0.060 | 0.267 | 1.000 | N | 2.866 | 0.257 | 1.140 | 4.264 |
| O | 0.149 | 0.573 | 0.279 | 1.000 | O | 0.635 | 2.441 | 1.188 | 4.264 |

TABLE 3

| | Actives to Moles (in 100 gram formula) | | | | | Moles to Molar Ration | | | |
|---|---|---|---|---|---|---|---|---|---|
| | CTAC | CAPB | CMEA | Total | | CTAC | CAPB | CMEA | Total |
| A | 0.002851 | 0.007917 | 0.002635 | 0.01340 | A | 0.2127 | 0.5907 | 0.1966 | 1.000 |
| B | 0.009496 | 0.002319 | 0.001772 | 0.01359 | B | 0.6989 | 0.1707 | 0.1304 | 1.000 |
| C | 0.007627 | 0.002859 | 0.003470 | 0.01396 | C | 0.5465 | 0.2049 | 0.2486 | 1.000 |
| D | 0.004064 | 0.005885 | 0.003899 | 0.01396 | D | 0.2935 | 0.4250 | 0.2816 | 1.000 |
| E | 0.005686 | 0.003534 | 0.005072 | 0.01429 | E | 0.3978 | 0.2473 | 0.3549 | 1.000 |
| F | 0.00019 | 0.011990 | 0.000632 | 0.01264 | F | 0.0015 | 0.9485 | 0.0500 | 1.000 |
| G | 0.005152 | 0.006753 | 0.001247 | 0.01315 | G | 0.3917 | 0.5134 | 0.0948 | 1.000 |
| H | 0.001263 | 0.009921 | 0.001904 | 0.01309 | H | 0.0965 | 0.7580 | 0.1455 | 1.000 |
| I | 0.008550 | 0.003920 | 0.000764 | 0.01323 | I | 0.6461 | 0.2962 | 0.0577 | 1.000 |
| J | 0.003677 | 0.008842 | 0.000249 | 0.01277 | J | 0.2880 | 0.6925 | 0.0195 | 1.000 |
| K | 0.000317 | 0.009137 | 0.004252 | 0.01371 | K | 0.0231 | 0.6667 | 0.3102 | 1.000 |
| L | 0.006522 | 0.004740 | 0.002277 | 0.01354 | L | 0.4817 | 0.3501 | 0.1682 | 1.000 |
| M | 0.007295 | 0.005625 | 0.000016 | 0.01294 | M | 0.5639 | 0.4348 | 0.0012 | 1.000 |
| N | 0.008957 | 0.000751 | 0.004686 | 0.01439 | N | 0.6223 | 0.0522 | 0.3256 | 1.000 |
| O | 0.001983 | 0.007132 | 0.004881 | 0.01400 | O | 0.1417 | 0.5096 | 0.3487 | 1.000 |

All formulations were created as aqueous surfactant systems, and the pH of each was adjusted to 4.8 with citric acid. An example of a formulation is shown in Table 4 for system “A.”

TABLE 4

| | |
|---|---|
| Formula Identifier | A |

*heat to 125-135 F. (52-57 C.). Remove from heat after CMEA dissolution

| Ingredient | Wt % | Amt. to Add (g) |
|---|---|---|
| Water | 86.95 | 869.53 |
| Cocamide MEA | 0.65 | 6.47 |
| CTAC (Varisoft 300) | 3.07 | 30.67 |
| Cocamidopropyl betaine | 6.83 | 68.31 |
| (40%) | | |

*adjust to pH = 4.8 with Citrosol 501 (50% Citric acid solution)

| | | |
|---|---|---|
| Water HOLE | 2.50 | 25.00 |
| Total | 100.00 | 1000.00 |

Prior art system (P) was created as a benchmark representing the surfactant system of a commercially available antibacterial hand soap that is used to gauge improvements to zein score for inventive systems A-O. Table 5, below, was used to formulate the benchmark system P.

TABLE 5

| | |
|---|---|
| Formula Identifier | Benchmark system P |

*heat to 125-135° F. (52-57° C.). Remove from heat after CMEA dissolution

| Ingredient | Wt % | Amt. to Add (g) |
|---|---|---|
| Water | 83.80 | 838.00 |
| CTAC (Varisoft 300) | 5.00 | 50.00 |
| Ammonyx LO | 3.25 | 32.50 |
| Ammonyx LMDO | 5.45 | 54.50 |

*adjust to pH = 4.8 with Citrosol 501

| | | |
|---|---|---|
| Water HOLE | 2.50 | 25.00 |
| Total | 100.00 | 1000.00 |

The ingredient activity for benchmark system P is CTAC=30% active, Ammonyx LO=30% active, and Ammonyx LMDO=33% active. In some instances, similar benchmark systems can include CTAC in an amount ranging from 0 to 7 wt %, Ammonyx LO in an amount ranging from 0 to 5 wt %, Ammonyx LMDO in an amount ranging from 0 to 7%, and the balance as water.

For each surfactant-only system, viscosity and zein were measured (the surfactant-only systems represent the “no salts” condition).

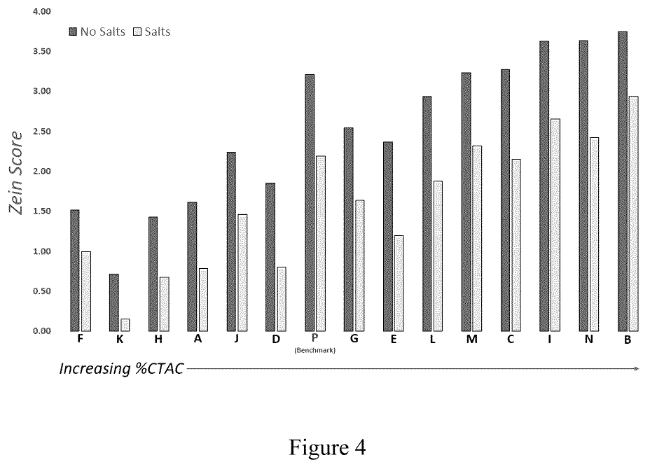

Next, the following materials were added to each system representing the “with salts” condition: 0.05 wt % active zinc sulfate, 0.05 wt % active Na-EDDS, 0.40 wt % active sodium benzoate, and 0.35 wt % active Glucamate DOE-120 (PEG-120 methyl glucose dioleate thickening polymer from Lubrizol). Viscosity and zein were measured again. Table 6 discloses the viscosity and zein score in descending order by % CTAC. A bar graph representation can be seen in FIG. 4. In some instances, systems as described in this paragraph and Table 6 can have a range of individual components representing the “with salts” condition as follows: 0 to 0.1 wt % active zinc sulfate, 0 to 0.1 wt % active Na-EDDS, 0 to 1.0 wt % active sodium benzoate, and 0 to 1.0 wt % active Glucamate DOE-120.

TABLE 6

| | Viscosity (cps) | | Zein Score | | % Active |
|---|---|---|---|---|---|
| ID | No Salts | With Salts | No Salts | With Salts | CTAC |
| F | 1 | 198 | 1.52 | 1.00 | 0.006 |
| K | 9 | 1210 | 0.72 | 0.16 | 0.101 |
| H | 3 | 4 | 1.43 | 0.68 | 0.404 |
| A | 3 | 1145 | 1.62 | 0.79 | 0.912 |
| J | 3 | 3 | 2.24 | 1.46 | 1.177 |
| D | 5 | 9630 | 1.86 | 0.81 | 1.300 |
| P | 3 | 4 | 3.21 | 2.19 | 1.500 |
| G | 4 | 4 | 2.55 | 1.64 | 1.649 |
| E | 4 | 13200 | 2.37 | 1.20 | 1.820 |
| L | 3 | 150 | 2.94 | 1.88 | 2.087 |
| M | 3 | 3 | 3.24 | 2.32 | 2.334 |
| C | 3 | 1030 | 3.28 | 2.15 | 2.441 |
| I | 3 | 3 | 3.63 | 2.66 | 2.736 |
| N | 3 | 990 | 3.64 | 2.43 | 2.866 |
| B | 3 | 3 | 3.75 | 2.94 | 3.039 |

The above data demonstrates that the ‘No Salt’ systems are unable to build viscosity on their own. However, by adding the salts/polymer, we can see that several of the specific surfactant ratio systems build viscosity while concomitantly lowering Zein score. Even instances where the salts do not help build viscosity, the resulting soap compositions exhibit markedly lower Zein scores. In view of the data above, examples B, F, G, H, I, J, M and N exhibit viscosities suitable for use as liquid soap compositions. Also in view of the data above, examples A, C, K and N exhibit viscosities suitable for use as gel or gel like soap compositions. In some instances, examples D and E may find utility as viscoelastic or substantially solid soap compositions.

Statistical software, such as from JMP Statistical Discovery LLC, can be used to analyze the viscosity data (with salts), the following results in Table 7 were obtained.

TABLE 7
Summary of Fit

| | |
|---|---|
| R Square | 0.940574 |
| R Square Adj | 0.889638 |
| Root Mean Square Error | 1358.993 |
| Mean of Response | 1969.5 |
| Observations (Or Sum Wgts) | 14 |

Parameter Estimates

| Term | Estimate | Std Error | t Ratio | Prob > |
|---|---|---|---|---|
| CMEA (Mixture) | 285717.12 | 44684.88 | 6.39 | 0.0004 |
| CAPB*CMEA | −390657.9 | 63077.12 | −6.19 | 0.0004 |
| CTACT*CMEA | −440746.1 | 75514.68 | −5.84 | 0.0006 |
| CTAC*CAPB*CMEA | 445770.49 | 77548.57 | 5.75 | 0.0007 |
| CAPB (Mixture) | 4250.9823 | 1808.471 | 2.35 | 0.0510 |
| CTAC*CAPB | −25635.93 | 12814.44 | −2.00 | 0.0855 |
| CTAC(Mixture) | 8197.9748 | 4899.594 | 1.67 | 0.1382 |

Tests on mixture main effects are confounded with the rest of the mixture

The R² of ˜94% for this model indicates a high degree of predictability. Additionally, several factors/factor combinations were found to be highly statistically significant, which is indicated by the p-value<0.05. Of particular significant is the high statistical significance of the CTAC*CAPB*CMEA three-way interaction. The “estimate” value of 445,770.49 represents the coefficient in the predictive equation. The high magnitude and positive sign for this value indicates a surprising and unexpected synergy between all three ingredients to effectively increase viscosity. This is especially surprising given the negative coefficients for the CAPB*CMEA and CTAC*CMEA, indicating that these two-way interactions drive viscosity lower. All three ingredients together, however, drive viscosity higher.

Furthermore, if you create a ternary plot using the mixture Profiler in the JMP software using the “With Salts” viscosity data, a ternary plot as seen in FIG. 1 is obtained.

The red line in FIG. 2 highlights an apparent minimum required % CMEA to achieve at least 3000 cps viscosity. This minimum viscosity level represents a threshold for consumer acceptability for a viscous (gel-type) antibacterial hand soap.

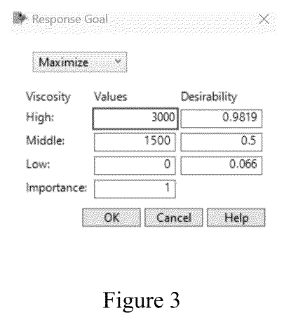

Additionally, by using the “Response Optimizer” tool (settings, shown in FIG. 3) in the JMP software, it can be determined that this minimum % CMEA corresponds to 0.157 (or 15.7% of the DOE compositional space) or 0.669% active CMEA. The “Response Optimizer was configured to maximize the response (viscosity) as close as possible to 3000 cps. The results are seen below in Table 8.

TABLE 8

| DOE | | | | | | | | |
|---|---|---|---|---|---|---|---|---|
| Ratio % | DOE Ratio % | | Predicted | Corresponding % Actives | | | | |
| (Fixed) | (optimized) | | Viscosity | | | | | Total % |
| CMEA | CTAC | CAPB | (CPS) | Desirability | CMEA | CTAC | CAPB | Actives |
| 0.150 | 0.382 | 0.468 | 2670 | 87.50% | 0.640 | 1.629 | 1.996 | 4.264 |
| 0.151 | 0.381 | 0.468 | 2723 | 89.24% | 0.644 | 1.625 | 1.996 | 4.264 |
| 0.152 | 0.380 | 0.468 | 2777 | 90.98% | 0.648 | 1.620 | 1.996 | 4.264 |
| 0.153 | 0.380 | 0.467 | 2831 | 92.74% | 0.652 | 1.620 | 1.991 | 4.264 |
| 0.154 | 0.379 | 0.467 | 2885 | 94.50% | 0.657 | 1.616 | 1.991 | 4.264 |
| 0.155 | 0.379 | 0.466 | 2940 | 96.28% | 0.661 | 1.616 | 1.987 | 4.264 |
| 0.156 | 0.378 | 0.466 | 2996 | 98.07% | 0.665 | 1.612 | 1.987 | 4.264 |
| 0.157 | 0.377 | 0.466 | 3052 | 99.06% | 0.669 | 1.608 | 1.987 | 4.264 |
| 0.158 | 0.377 | 0.465 | 3108 | 99.38% | 0.674 | 1.608 | 1.983 | 4.264 |

### Zein Score Analysis

Prior to measuring Zein score the above surfactant systems were diluted (25% water+75% formula) to approximate the surfactant concentration of an actual commercial hand soap during use. These following results are therefore expected to be consumer relevant in terms of mildness or harshness to the skin.

### No Salts

Statistical analysis of the zein score data of the no salts is recorded below in Table 9.

TABLE 9
Summary of Fit

| | |
|---|---|
| R Square | 0.995431 |
| R Square Adj | 0.991516 |
| Root Mean Square Error | 0.088609 |
| Mean of Response | 2.485 |
| Observations (Or Sum Wgts) | 14 |

Parameter Estimates

| Term | Estimate | Std Error | t Ratio | Prob > |
|---|---|---|---|---|
| CTAC (Mixture) | 5.2231493 | 0.319462 | 16.35 | <0.0001 |
| CAPB (Mixture) | 0.6546593 | 0.117915 | 14.03 | <0.0001 |
| CTAC*CAPB | −1.522555 | 0.835523 | −1.82 | 0.1112 |
| CAPB*CMEA | −2.115202 | 4.112735 | −0.51 | 0.6229 |
| CTAC*CMEA | 2.3458908 | 4.923685 | 0.48 | 0.6483 |
| CTAC*CAPB*CMEA | 2.1380838 | 5.056293 | 0.42 | 0.6851 |
| CMEA (Mixture) | −1.124594 | 2.91353 | −0.39 | 0.7110 |

Tests on mixture main effects are confounded with the rest of the mixture

The R² of ˜99.5% suggests a high degree of predictability for this model. Additionally, the statistical significance for CTAC and CAPB indicates that these surfactants by themselves drive zein score up signifying an increase in harshness on the skin.

Statistical analysis of the zein score data of the no salts is recorded below in Table 10.

TABLE 10
Salts
Summary of Fit

| | |
|---|---|
| R Square | 0.995891 |
| R Square Adj | 0.992368 |
| Root Mean Square Error | 0.073658 |
| Mean of Response | 1.58 |
| Observations (Or Sum Wgts) | 14 |

Parameter Estimates

| Term | Estimate | Std Error | t Ratio | Prob > |
|---|---|---|---|---|
| CTAC (Mixture) | 4.8920324 | 0.265561 | 18.42 | <0.0001 |
| CAPB (Mixture) | 1.1401539 | 0.09802 | 11.63 | <0.0001 |
| CTAC*CAPB | −3.424425 | 0.69455 | −4.93 | 0.0017 |
| CMEA (Mixture) | −2.385186 | 2.421949 | −0.98 | 0.3575 |
| CTAC*CAPB*CMEA | 1.7003776 | 4.203181 | 0.40 | 0.6979 |
| CAPB*CMEA | −1.219883 | 3.41882 | −0.36 | 0.7317 |
| CTAC*CMEA | −0.611723 | 4.092943 | −0.15 | 0.8554 |

Tests on mixture main effects are confounded with the rest of the mixture

The R² of ˜99.5% suggests a high degree of predictability for this model. Again, CTAC and CAPB by themselves were found to be statistically significant and increase the zein score. However, what is surprising is that by incorporating salt, a new synergy is found between CTAC*CAPB that drives zein score down (milder). This effect is confirmed by the p-value of 0.0017 (statistically significant) and the negative coefficient of −3.424425.

Furthermore, the data plot of the zein score showcases the benefits of adding salts. For every system, the zein score decreases (becomes milder) as salt is added. As % CTAC increases, the zein score increases (becomes harsher). However, when comparing the data points to the benchmark ‘P’, many of the DOE surfactant systems produce a lower zein score (both with and without salt), even at higher % CTAC scores. Two of the surfactants in the benchmark system (lauramine oxide and lauryl/myristyl amidoamine oxide) can be replaced with two new surfactants (CAPB and CMEA) to achieve better zein scores, taking advantage of the newly discovered synergy between CAPB and CMEA to lower zein score and enabling a higher inclusion of CTAC. A higher % CTAC can drive increased antibacterial efficacy.

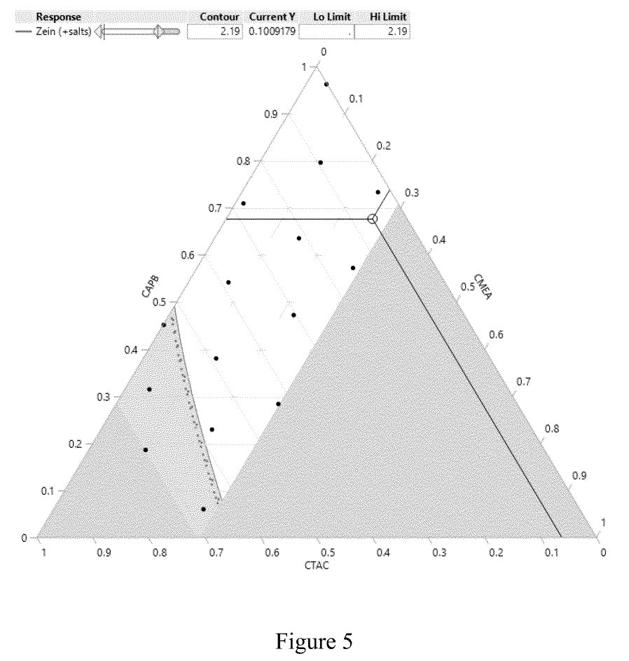

A ternary plot was created using the Mixture Profiler in the JMP software for the ‘With Salts’ data as displayed in FIG. 5. The ‘With Salts’ data was chosen as this more closely represents a commercially viable formula. Note that the “bullseye” with three connecting lines was placed at an arbitrary point.

In the plot shown in FIG. 5, the red/shaded region indicates compositions with higher zein scores (harsher) than the benchmark system P. The white region indicates the range of CTAC/CAPB/CMEA blends that yield lower zein scores (milder) than the benchmark formulation.

The thickening polymer, DOE-120, was added to increase the viscosity of the soap composition. Select example soap compositions were therefore assessed for zein score to ensure that the zein score reductions resulted from the addition of salt and not the addition of polymer Table 12. These formulations were chosen due to their differences in CTAC/CAPB/CMEA ratios and representation of the overall DOE data set range. In Table 12, the data provided for each sample is as below:
No Salts=Zein Score for surfactant only, pH 4.8
DOE-120=Zein Score for surfactant+0.35% DOE-120
With Salts and DOE=Zein Score for surfactant+0.05% zinc sulfate+0.05% Na-EDDS+0.040% sodium benzoate, pH 4.8
With Salts and DOE=Zein Score for surfactant+0.35% DOE-120+0.05% zinc sulfate+0.05% Na-EDDS+0.040% sodium benzoate, pH 4.8
% Reduction was calculated as a percentage of the total available zein to dissolve.

% Reduction=(Zein score difference/Total Zein)*100%

TABLE 12

| | No | With DOE-120 | With Salts | With Salts and DOE-120 |
|---|---|---|---|---|
| ID | Salts | (% Reduction) | (% Reduction) | (% Reduction) |
| B | 3.75 | 3.56 (4.75%) | 2.86 (22.25%) | 2.94 (20.51%) |
| E | 2.37 | 2.37 (0.00%) | 1.28 (27.25%) | 1.20 (29.62%) |
| F | 1.52 | 1.41 (2.75%) | 1.05 (11.75%) | 1.00 (13.16%) |
| N | 3.64 | 3.57 (1.75%) | 2.56 (27.00%) | 2.43 (30.63%) |

The addition of the DOE-120 polymer only to the surfactant system showed little-to-no effects in terms of lowering the zein score. However, when adding the salts, there is a dramatic reduction to the zein score. The final column of Table 12 includes the DOE-120 polymer and the salts. As can be seen from this data, the sequential addition of DOE-120 and the salts does not result in an additive reduction in Zein score, which further supports the conclusion that the reduction in zein score is driven by the addition of counterions (anions) of the salts and not by the addition of the DOE-120 polymer.

Lastly, an additional zein score was obtained for system ‘E’ at a greater dilution (75% water+25% formula) to show that the effects of zein score reduction through salt addition is independent of surfactant concentration. This system was chosen arbitrarily from the possible systems. The results can be found in Table 13.

TABLE 13

| | 25% formula + 75% | | | 75% formula + 25% | | |
|---|---|---|---|---|---|---|
| | water | | % | water | | % |
| ID | No salt | With Salt | Reduction | No Salt | With Salt | Reduction |
| E | 1.00 | 0.37 | 15.75% | 2.23 | 1.33 | 22.50% |

While certain implementations have been described in terms of what may be considered to be specific aspects, the present disclosure is not limited to the disclosed aspects. Additional modifications and improvements to the aforementioned synthetic scents and the approaches by which they are incorporated into instructional and assessment phases of curricula may be apparent to those skilled in the art. Moreover, the many features and advantages of the disclosure are apparent from the detailed specification, and thus, it is intended by the appended claims to cover all such features and advantages of the present disclosure which fall within the spirit and scope of the disclosure.

1. A soap composition comprising:
at least one non-ionic surfactant;
at least one cationic surfactant;
at least one amphoteric or zwitterionic surfactant; and
at least one salt in an amount from about 0.01 wt % to about 1.0 wt % of the soap composition.

2. The soap composition of claim 1, wherein the soap composition has a cationic surfactant(s): amphoteric/zwitterionic surfactant(s): non-ionic surfactant(s) molar percentage (mol %) ratio ranging from 0.1-75.0:5.0-99.0:0.1-40.0.

3. The soap composition of claim 1, wherein the at least one non-ionic surfactant is an alkanolamide.

4. The soap composition of claim 3, wherein the alkanolamide is cocamide monoethanolamine (CMEA).

5. The soap composition of claim 1, wherein the at least one cationic surfactant is an ammonium salt.

6. The soap composition of claim 5, wherein the ammonium salt is a quaternary ammonium salt.

7. The soap composition of claim 6, wherein the quaternary ammonium salt is cetyl trimethyl ammonium chloride (CTAC).

8. The soap composition of claim 1, wherein the at least one amphoteric or zwitterionic surfactant is a betaine.

9. The soap composition of claim 8, wherein the betaine is cocamidopropyl betaine (CAPB).

10. The soap composition of claim 1, wherein the at least one salt is sodium benzoate, zinc sulfate, sodium ethylenediamine-N,N′-disuccinate (Na-EDDS), or any combination thereof.

11. The soap composition of claim 1, wherein the soap composition has a viscosity ranging from about 1 cps to about 6,000 cps.

12. The soap composition of claim 1, wherein the soap composition has a viscosity ranging from about 1,000 cps to about 3,000 cps.

13. The soap composition of claim 1, wherein the soap composition has a viscosity ranging from about 1 cps to about 1,000 cps.

14. The soap composition of claim 1, wherein the soap composition has a Zein score of less than 3.0%.

15. The soap composition of claim 1, wherein the soap composition has a Zein score of less than 2.0%.

16. The soap composition of claim 1, wherein the soap composition has a Zein score of less than 1.0%.

17. The soap composition of claim 1, wherein the soap composition has a pH ranging from about 2 to about 7 or from about 7 to about 10.

18. The soap composition of claim 1, wherein the soap composition further comprises a rheology modifier.

19. The soap composition of claim 18, wherein the rheology modifier is a thickener.

20. The soap composition of claim 1, wherein the soap composition is a hand soap composition.
